(19) United States
(12) Patent Application Publication
(10) Pub. No.: US 2026/0245746 A1
(43) Pub. Date: Aug. 20, 2026

(54) DRUG INFORMATION PROCESSING METHOD, APPARATUS, DEVICE AND MEDIUM

(71) Applicant: Beijing Zitiao Network Technology Co., Ltd., Beijing (CN)

(72) Inventors: Jiajing LI, Beijing (CN); Zhili XIONG, Beijing (CN); Lin YANG, Beijing (CN)

(21) Appl. No.: 19/544,460

(22) Filed: Feb. 19, 2026

(30) Foreign Application Priority Data
Feb. 19, 2025 (CN) 202510186760.4

(51) Int. Cl. G16H 70/40

(52) U.S. Cl. CPC G16H 70/40

(57) ABSTRACT

Embodiments of the present disclosure provide a resource information processing method and apparatus, an electronic device, and a storage medium. The method includes: acquiring user input content; performing search in a drug knowledge base based on the user input content to obtain a search result; determining a target corresponding to the user input content based on the user input content; determining a prompt based on the search result and the target, and inputting the prompt into a pre-trained drug processing model to obtain an output result corresponding to the user input content; and displaying the output result. With the above technical solution, by using a search enhanced generation technology, a user may obtain a more accurate output result without having high professional knowledge, which significantly improves the convenience of information query.

### CROSS-REFERENCE TO RELATED APPLICATION

This application claims the priority to and benefits of the Chinese Patent Application, No. 202510186760.4, which was filed on Feb. 19, 2025. The aforementioned patent application is hereby incorporated by reference in its entirety.

### TECHNICAL FIELD

The present disclosure relates to a drug information processing method and apparatus, an electronic device, and a storage medium.

### BACKGROUND

Users have the demands for querying and understanding the relevant knowledge of drugs, consulting the usage, dosage, indications and other drug popular science and medication guidance. At present, users usually obtain relevant information by actively searching for the generic name of a drug or directly viewing the instructions in the medicine box, which requires a high degree of professionalism from users and is low in information query convenience. In the related art, the general model can require about the content related to drugs, but the general model is prone to hallucinations, and the accuracy of the result is low, so it is difficult to meet the requirements for accuracy and convenience at the same time.

### SUMMARY

The embodiment of the present disclosure provides a drug information processing method and apparatus, an electronic device, and a non-transitory computer-readable storage medium.

At least one embodiment of the present disclosure provides a drug information processing method, and the method includes:

acquiring user input content;

performing search in a drug knowledge base based on the user input content to obtain a search result;

determining a target corresponding to the user input content based on the user input content;

determining a prompt based on the search result and the target, and inputting the prompt into a pre-trained drug processing model to obtain an output result corresponding to the user input content; and

displaying the output result.

At least one embodiment of the present disclosure further provides a drug information processing apparatus, and the apparatus includes:

a first acquisition module, configured to acquire user input content;

a search module, configured to perform search in a drug knowledge base based on the user input content to obtain a search result;

a determination module, configured to determine a target corresponding to the user input content based on the user input content;

an input module, configured to determine a prompt based on the search result and the target, and input the prompt into a pre-trained drug processing model to obtain an output result corresponding to the user input content; and

a first display module, configured to display the output result.

At least one embodiment of the present disclosure further provides an electronic device, and the electronic device includes: a processor; and a memory configured to store executable instructions that may be executed by the processor, where the processor is configured to read the executable instructions from the memory, and execute the executable instructions to implement the drug information processing method provided by the embodiments of the present disclosure.

At least one embodiment of the present disclosure further provides a non-transitory computer-readable storage medium. The storage medium stores a computer program, where the computer program is used to perform the drug information processing method provided by the embodiments of the present disclosure.

Compared with the prior art, the technical solutions provided by the embodiments of the present disclosure have the following advantages. The drug information processing method provided by the embodiments of the present disclosure includes: acquiring user input content; performing search in a drug knowledge base based on the user input content to obtain a search result; determining a target corresponding to the user input content based on the user input content; determining a prompt based on the search result and the target, and inputting the prompt into a pre-trained drug processing model to obtain an output result corresponding to the user input content; and displaying the output result. With the above technical solution, for the acquired user input content, the corresponding search result is retrieved from the drug knowledge base, and the target is determined according to the user input content. Then, the pre-trained drug processing model is used to process the prompt determined based on the search result and the target to obtain the target output result. By using the search enhanced generation technology, the user may obtain a more accurate target output result without having high professional knowledge, which significantly improves the convenience of information query. Moreover, target recognition helps to accurately match user needs and generate an output result that conforms to the user target, thereby effectively improving the accuracy of drug information query.

### BRIEF DESCRIPTION OF DRAWINGS

In order to more clearly explain the technical solutions in the embodiments of the present disclosure, the drawings required to be used in the description of the embodiments will be briefly introduced below. Obviously, the drawings in the following description are some embodiments of the present disclosure, and for those skilled in the art, other drawings may be obtained according to these drawings without paying creative efforts.

FIG. 1 is a schematic flowchart of a drug information processing method provided by at least one embodiment of the present disclosure;

FIG. 2 is a schematic diagram of a drug associated page provided by at least one embodiment of the present disclosure;

FIG. 3 is a schematic diagram of a first drug information page provided by at least one embodiment of the present disclosure;

FIG. 4 is a schematic diagram of a second drug information page provided by at least one embodiment of the present disclosure;

FIG. 5 is a schematic diagram of a third drug information page provided by at least one embodiment of the present disclosure;

FIG. 6 is a schematic diagram of a fourth drug information page provided by at least one embodiment of the present disclosure;

FIG. 7 is a schematic diagram of a fifth drug information page provided by at least one embodiment of the present disclosure;

FIG. 8 is a schematic diagram of a sixth drug information page provided by at least one embodiment of the present disclosure;

FIG. 9 is a schematic structural diagram of a drug information processing apparatus provided by at least one embodiment of the present disclosure; and

FIG. 10 is a schematic structural diagram of an electronic device provided by at least one embodiment of the present disclosure.

### DETAILED DESCRIPTION

Hereinafter, embodiments of the present disclosure will be described in more detail with reference to the drawings. Although certain embodiments of the present disclosure are shown in the drawings, it should be understood that the present disclosure may be implemented in various forms and should not be construed as being limited to the embodiments set forth herein. Rather, these embodiments are provided for a more thorough and complete understanding of the present disclosure. It should be understood that the drawings and embodiments of the present disclosure are only for illustrative purposes, and are not intended to limit the scope of protection of the present disclosure.

It should be understood that various steps described in method implementations of the present disclosure may be performed in different orders, and/or performed in parallel. Furthermore, the method implementations may include additional steps and/or omit the performance of the illustrated steps. The scope of the present disclosure is not limited in this regard.

As used herein, the term "include/comprise" and variations thereof are open-ended inclusions, that is, "include/comprise but not limited to". The term "based on" is "at least partially based on". The term "an embodiment" means "at least one embodiment"; the term "another embodiment" means "at least one additional embodiment"; and the term "some embodiments" means "at least some embodiments". Relevant definitions of other terms will be given in the following description.

It should be noted that concepts such as "first" and "second" mentioned in the present disclosure are only used to distinguish different apparatuses, modules or units, and are not used to limit the order or interdependence of the functions performed by these apparatuses, modules or units.

It should be noted that the modifiers "a" and "a plurality of" mentioned in the present disclosure are illustrative and not restrictive, and those skilled in the art should understand that unless otherwise clearly indicated in the context, they should be understood as "one or more".

The names of messages or information exchanged between a plurality of apparatuses in the implementations of the present disclosure are only used for illustration, and are not used to limit the scope of these messages or information.

The traditional drug knowledge base has many limitations in the use process. First of all, the traditional drug knowledge base requires users to have high professional knowledge and information judgment ability to accurately locate related problems, which is difficult for ordinary users. Secondly, professional content is usually long and not convenient for quick query, and users need to read the full text to find the required information. In addition, for the generic name of a drug, products from different manufacturers may have differences, and traditional popular science cannot accurately answer questions with finer granularity. Directly relying on the general model to provide answers is prone to model hallucinations, resulting in inaccurate results. In summary, it is difficult for the above methods to take into account both convenience and information accuracy, and thus cannot fully meet user’s needs.

In order to solve the above problems, at least one embodiment of the present disclosure provides a drug information processing method. The method will be described below in combination with specific embodiments.

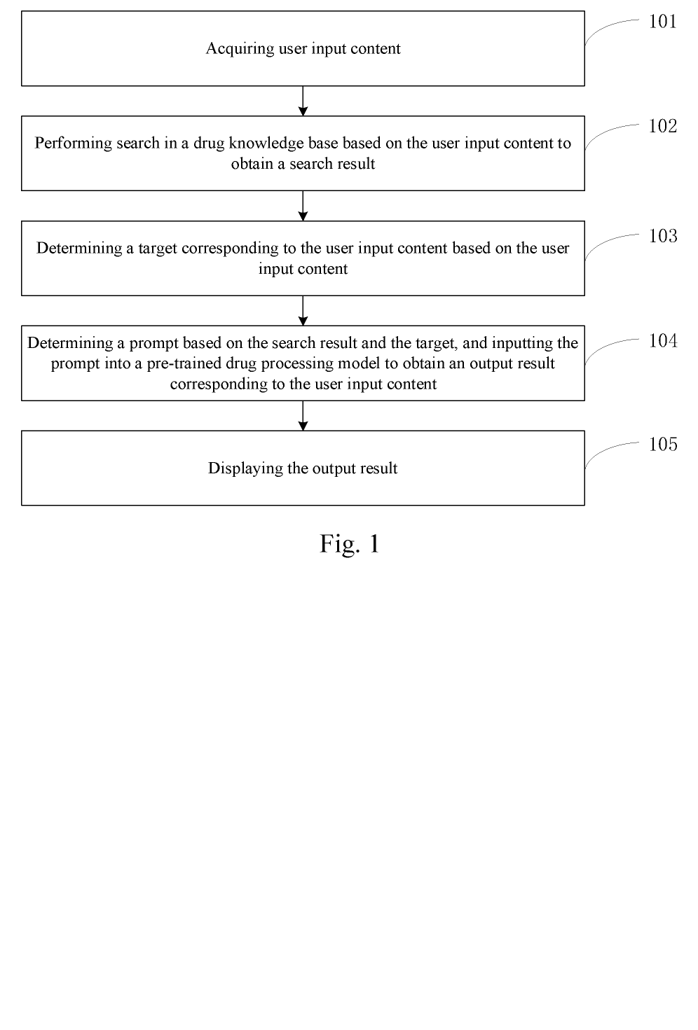

FIG. 1 is a schematic flowchart of a drug information processing method provided by at least one embodiment of the present disclosure. The method may be performed by a drug information processing apparatus, where the apparatus may be implemented by software and/or hardware, and may generally be integrated in an electronic device. As shown in FIG. 1, the method includes the following steps.

Step 101: acquiring user input content.

The drug information processing method provided by the present embodiment of the present disclosure is implemented based on a drug information processing system. Specifically, the drug information processing system refers to a system that provides users with drug popular science and medication guidance such as querying and understanding the relevant knowledge of drugs, and consulting the usage, dosage, indications of drugs, etc. For example, the system provides users with querying and understanding the relevant knowledge of drugs in the form of questions and answers.

The user input content refers to information input by a user through an interface (such as a web page, an application program, or other software interfaces), and the form of the user input content is not limited. Specifically, the user input content may be a question related to any drug, such as a question about the efficacy, usage, dosage, etc., of drug A. After receiving an information processing request of the user for a first drug, the drug information processing apparatus parses the processing request to acquire the user input content.

Exemplarily, the acquiring user input content includes at least one selected from the group consisting of: acquiring the user input content in response to an input operation in a search box; acquiring a first drug image in response to a trigger operation on a photographing recognition control, and obtaining the user input content based on recognizing the first drug image; and acquiring the user input content in response to a trigger operation on a drug consultation entry control in a drug associated page.

In an optional implementation, the user input content is acquired in response to an input operation in a search box.

The search box refers to a control for inputting query content. Generally, the search box includes an input box and a search button. The input box is an area where a user inputs keywords or query sentences, such as a rectangular text box, in which the user may input the content to be searched. The search button refers to a control for triggering a search function. After the user finishes inputting the search content, clicking the search button may trigger the search function. The input operation refers to a process in which the user inputs information to the drug information processing apparatus through various input devices, such as a keyboard, a mouse, a touch screen, a microphone, a stylus, etc.

In this embodiment of the present disclosure, after receiving the input operation of the user in the search box, the drug information processing apparatus parses the input operation to acquire the user input content.

In another implementation, a first drug image is acquired in response to a trigger operation on a photographing recognition control, and the user input content is obtained based on recognition of the first drug image.

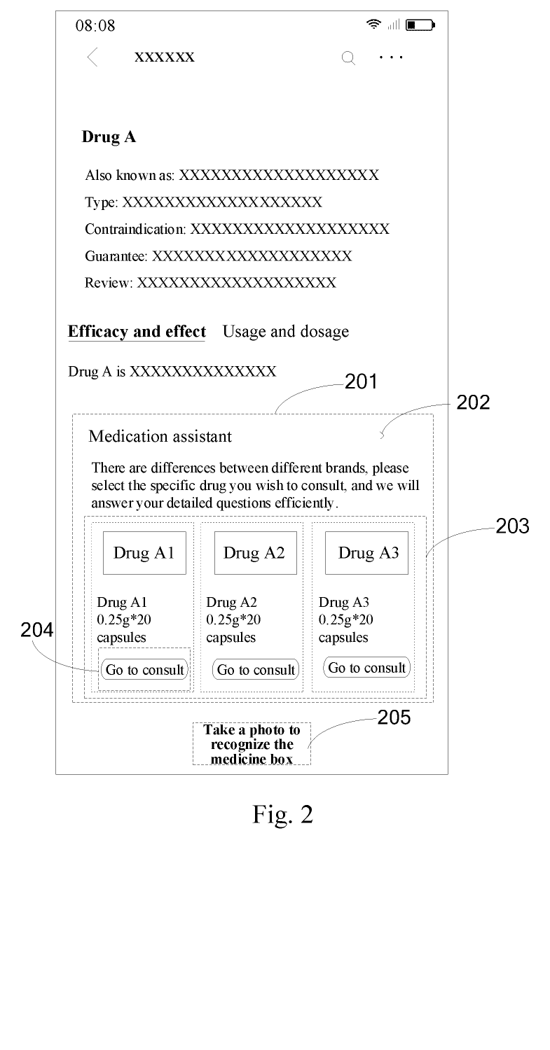

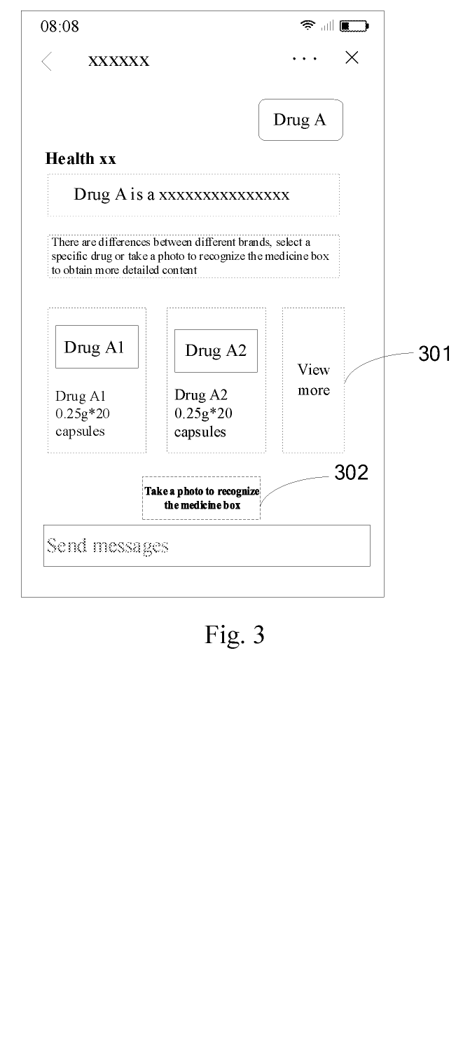

The photographing recognition control refers to a functional component that allows the user to take a photo through a camera or select a picture from an album. Specifically, the photographing recognition control may be displayed below the drug consultation entry control in the drug associated page, as shown in FIG. 2, FIG. 2 is a schematic diagram of a drug associated page provided by at least one embodiment of the present disclosure. An area 205 in FIG. 2 displays the photographing recognition control. The photographing recognition control may also be displayed on the drug information page, as shown in FIG. 3, FIG. 3 is a schematic diagram of a first drug information page provided by at least one embodiment of the present disclosure. An area 302 in FIG. 3 displays the photographing recognition control. The trigger operation on the photographing recognition control may include a gesture operation on the photographing recognition control (such as clicking, long pressing, double-clicking, etc.), a voice control operation, an expression control operation, or the like. The first drug image refers to an image containing information and appearance features of a specific drug, which is selected by the user from the album or photographed by the user through the camera.

In this embodiment of the present disclosure, after receiving the trigger operation of the user on the photographing recognition control, the drug information processing apparatus invokes the album, acquires the first drug image after the user selects the first drug image, and recognizes the first drug image to obtain the user input content.

In another embodiment of the present disclosure, after receiving the trigger operation of the user on the photographing recognition control, the drug information processing apparatus invokes the camera, acquires the first drug image after the user photographs the first drug image, and recognizes the first drug image to obtain the user input content.

In another implementation, the user input content is acquired in response to a trigger operation on a drug consultation entry control in a drug associated page.

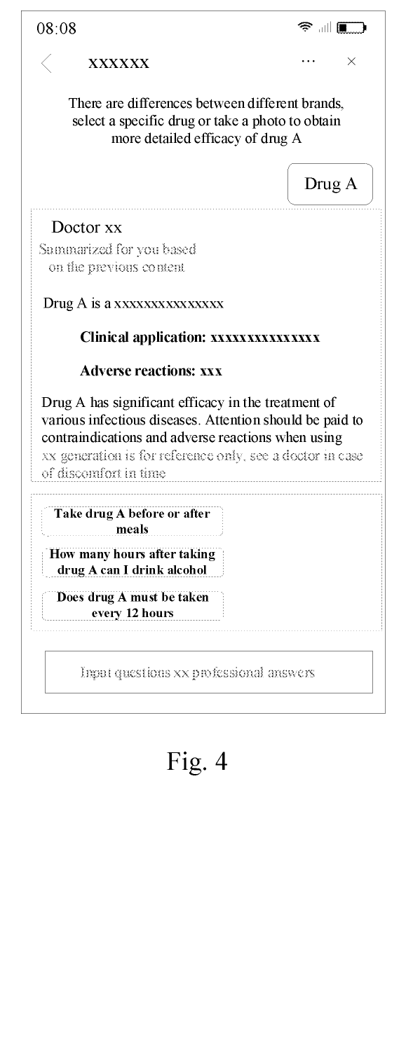

The drug associated page refers to a page design form that takes a drug as the core and integrates all kinds of information closely related to the drug on one page and presents the page to the user, which is a page for displaying drug-related information. The drug associated page includes drug information such as drug name and description, uses and functions, etc. For example, the drug associated page may be an encyclopedia page of a drug, or may be other pages that display drugs. The drug consultation entry control is a functional control for triggering entry into the drug information processing system, and the drug consultation entry control may be in any shape. The trigger operation on the drug consultation entry control may include a gesture operation on the drug consultation entry control (such as clicking, long pressing, double-clicking, etc.), a voice control operation, an expression control operation, or the like. As shown in FIG. 2, which is a drug associated page, an area 201 in FIG. 2 displays a drug consultation entry control, such as a drug consultation entry control 202, the drug consultation entry control 202 is used to trigger entry into a drug information page as shown in FIG. 4. FIG. 4 is a schematic diagram of a second drug information page provided by at least one embodiment of the present disclosure.

In this embodiment of the present disclosure, after receiving the trigger operation of the user on the drug consultation entry control in the drug associated page, the drug information processing apparatus determines a drug name corresponding to corresponding drug information in the drug associated page as the user input content.

The drug consultation entry control includes at least one piece of second drug information associated with first drug information displayed on the drug associated page. The acquiring the user input content in response to a trigger operation on a drug consultation entry control in a drug associated page includes: in response to a trigger position of the trigger operation on the drug consultation entry control being in a non-information position, determining a first drug name corresponding to the first drug information as the user input content; and in response to the trigger position of the trigger operation on the drug consultation entry control being in third drug information in the at least one piece of second drug information, determining a third drug name corresponding to the third drug information as the user input content.

In an optional implementation, if the trigger position of the trigger operation on the drug consultation entry control is in a non-information position, a first drug name corresponding to the first drug information is determined as the user input content.

The first drug information refers to information related to a first drug. The first drug refers to the drug currently displayed on the associated page. The second drug information refers to related information of a second drug associated with the first drug. Specifically, the second drug may be a drug of the same kind or similar drugs manufactured by a different manufacturer from the first drug. The non-information position refers to an area unrelated to the drug information, such as a blank position of the associated page or a position of the drug consultation entry control 202 shown in FIG. 2.

The first drug name refers to a drug name corresponding to the first drug information. The drug name is a specific title used to identify and distinguish different drugs. Specifically, the drug name may be a generic name, that is, the legal name of the drug. The drug name may also be a brand name, which is a name that a drug manufacturer uses to distinguish its own product from drugs with the same generic name produced by other manufacturers.

In this embodiment of the present disclosure, if the trigger position of the trigger operation on the drug consultation entry control is in the non-information position, after receiving the trigger operation of the user on the drug consultation entry control, the drug information processing apparatus determines the first drug name corresponding to the first drug information as the user input content.

Exemplarily, if the trigger position of the trigger operation on the drug consultation entry control is at the position of the drug consultation entry control 202 (i.e., the non-information position) as shown in FIG. 2, after receiving the trigger operation of the user on an area 202 as shown in FIG. 2, the drug information processing apparatus displays the drug information page as shown in FIG. 4, where the user input content in the drug information page is drug A (i.e., the first drug name), that is, the first drug name is determined as the user input content.

In another optional implementation, if the trigger position of the trigger operation on the drug consultation entry control is in third drug information in the at least one piece of second drug information, a third drug name corresponding to the third drug information is determined as the user input content.

The third drug information refers to any drug information in the at least one piece of second drug information. The trigger operation on the third drug information may include a gesture operation on the third drug information (such as clicking, long pressing, double-clicking, etc.), a voice control operation, an expression control operation, or the like. The third drug name refers to the name of the drug corresponding to the third drug information.

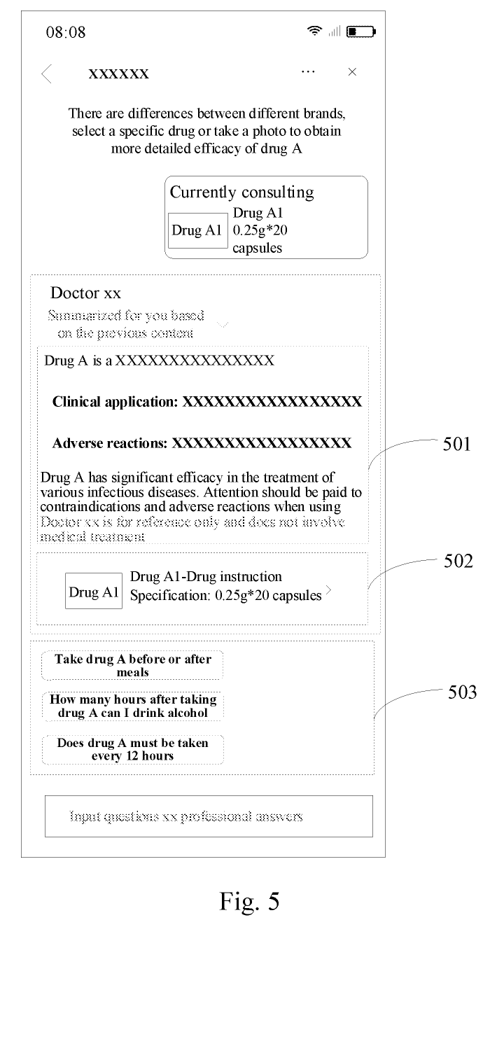

Continuing to refer to FIG. 2, as shown in FIG. 2, the first drug information is drug A, and the at least one piece of second drug information is drug A1, drug A2 and drug A3. Taking the drug A1 in the second drug information as the third drug information as an example, an area 203 in FIG. 2 displays the drug A1, the drug A2 and the drug A3 associated with the drug A, which for example may be drugs of the same kind or similar drugs manufactured by different manufacturers. If the trigger position of the trigger operation on the drug consultation entry control is in an area 204 (i.e., the third drug information), the drug A1 (i.e., the third drug name corresponding to the third drug information) is determined as the user input content. The area 204 is used to trigger entry into the drug information page as shown in FIG. 5. FIG. 5 is a schematic diagram of a third drug information page provided by at least one embodiment of the present disclosure.

In this embodiment of the present disclosure, if the trigger position of the trigger operation on the drug consultation entry control is in the third drug information in the at least one piece of second drug information, after receiving the trigger operation of the user on the third drug information, the drug information processing apparatus determines the third drug name corresponding to the third drug information as the user input content.

Exemplarily, if the trigger position of the trigger operation on the drug consultation entry control is in the area 204 (i.e., the third drug information), after receiving the trigger operation of the user on the area 204 as shown in FIG. 2, the drug information processing apparatus displays the drug information page as shown in FIG. 5, where the user input content in the drug information page is the drug A1 (i.e., the third drug name).

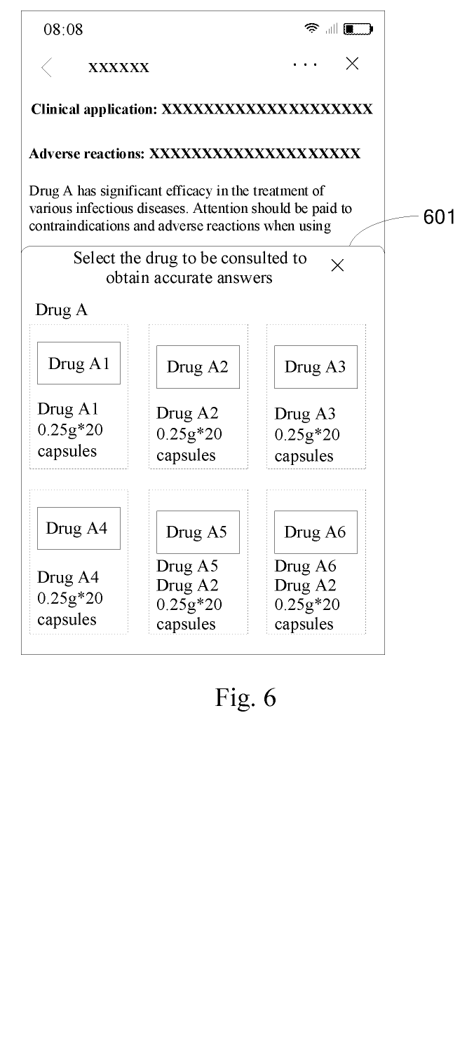

In another implementation, as shown in FIG. 3, the second drug information (i.e., the drug A1 and the drug A2) associated with the first drug information is displayed on the drug information page. When the user swipes to view the second drug information associated with the first drug information, a view more control 301 is displayed. The user may click the view more control 301 to display a pop-up layer 601 as shown in FIG. 6, where the pop-up layer 601 displays a plurality of pieces of second drug information to guide the user to select an accurate drug. FIG. 6 is a schematic diagram of a fourth drug information page provided by at least one embodiment of the present disclosure.

In the above solution, the drug information processing apparatus provides the user with an intuitive user interface, so that the user may input a drug question in various ways, such as the input operation in the search box, the trigger operation on the photographing recognition control, the trigger operation on the drug consultation entry control, etc., which improves the diversity and convenience of acquiring drug questions.

Step 102: performing search in a drug knowledge base based on the user input content to obtain a search result.

The drug knowledge base refers to a system or database that specifically stores, manages and provides drug-related knowledge. Specifically, the drug knowledge base may be constructed by digitalizing drug instructions and using the RAG (Retrieval-Augmented Generation) technology, so as to improve the convenience and accuracy of medication guidance and meet various query needs of users. The search result refers to drug information that matches the user input content and is retrieved from the drug knowledge base through query by the user input content.

In this embodiment of the present disclosure, the drug information processing apparatus may perform vectorization processing on the user input content to transform the user input content into a vector form, and search for drug information included in the drug knowledge base, for example, in a similarity matching manner, to obtain the search result.

Specifically, a method for constructing the drug knowledge base includes: acquiring drug information of a plurality of drugs, and constructing the drug knowledge base based on the drug information of the plurality of drugs, where the drug knowledge base includes structured data and unstructured data.

The drug information refers to various information about the drug itself and the characteristics and changes of the drug in the use process. Specifically, the drug information may include various information sources such as drug instructions, drug use experience, and drug databases. The drug instruction refers to a legal document provided by a drug manufacturer and containing important information of a drug, which is an important basis for guiding users to reasonably use the drug. The drug use experience refers to experience accumulated by users in terms of efficacy, feelings, problems, etc., in the actual use process of the drug after the drug is put on the market.

The structured data refers to data organized and stored according to pre-defined rules and formats. This kind of data has a clear structure and may usually be represented by a table (such as a table in a relational database), a document in a fixed format, etc. Taking the drug A as an example, the drug A includes multiple fields: generic name, manufacturer, indications, etc. Information of each field may be described in a paragraph, for example, the manufacturer of the drug A is "manufacturer a", and the indications are "treatment of respiratory tract infections", etc. The unstructured data refers to data that has no pre-defined structure or format, does not follow a fixed data model, and is difficult to store and manage in the form of a table in a traditional relational database. For example, if a piece of text has all its content connected together and lacks a clear segmentation, it belongs to unstructured data.

In this embodiment of the present disclosure, the drug information processing apparatus acquires drug information of a plurality of drugs, and integrates the drug information of the plurality of drugs. Specifically, different names of the same drug are unified into the generic name, and at the same time, duplicate drug information is removed. Then, the integrated drug information is vectorized and stored in the drug knowledge base to support fast search using a vector database.

In an optional implementation, the performing search in a drug knowledge base based on the user input content to obtain a search result includes: performing search in the drug knowledge base based on the user input content through a keyword search manner and a semantic search manner, to acquire a second drug name and a first drug content segment, and determine the second drug name and the first drug content segment as the search result.

The keyword search manner refers to a search method in which specific keywords (such as drug names, disease names, symptoms, or other related words) are input to search for content matching these keywords in the drug knowledge base. The semantic search manner refers to a search method in which, through correct analysis of the grammatical structure, accurate understanding of the meaning of words and the relationship between words, content semantically matching is searched for in the drug knowledge base at the semantic level, so as to use natural language processing technology to improve the accuracy of search, so that user targets may be understood and relevant content may be found. The first drug content segment refers to a part of specific information content related to the first drug. Specifically, the first drug content segment may be a part of the first drug instruction, such as usage and dosage.

In this embodiment of the present disclosure, after acquiring the user input content, the drug information processing apparatus first parses the user input content input by the user through the keyword search manner, extracts keywords in the user input content, performs matching in the drug knowledge base, and searches for a second drug name including the keywords and an information set related thereto in the drug instruction, the use experience and the drug database. Then, the drug information processing apparatus applies the semantic search manner to convert the user input content input by the user into a semantic vector, and uses natural language processing to recognize a semantically associated first drug content segment from the above information set.

Exemplarily, taking the user input content as "what is the contraindication of drug A", first, the user input of "what is the contraindication of drug A" is parsed through the keyword search manner, and keywords "drug A" and "contraindication" are extracted. The keywords "drug A" and "contraindication" are matched in the drug knowledge base, and an information set of "drug A" and related "contraindication" in the drug instruction, the use experience and the drug database is searched for. Then, the drug information processing apparatus applies the semantic search manner to convert the user input of "what is the contraindication of drug A" into a semantic vector, and uses natural language processing to recognize a semantically associated first drug content segment from the above information set.

In the above solution, the drug information processing apparatus may accurately extract the search result related to the user input content from the drug knowledge base. By combining the keyword search manner and the semantic search manner, it is ensured that the user may obtain a comprehensive, accurate and semantically matching search result.

Step 103: determining a target corresponding to the user input content based on the user input content.

The target refers to a purpose or need that a user expects to achieve after asking a question. According to different needs of users, the target may usually be divided into the following categories: an information target: the user expects to obtain an explanation or detailed information of a certain concept. For example, the user inquires about the use, ingredients or side effects of a certain drug. An operation target: the user expects to obtain guidance or specific steps. For example, the user inquires about the usage or dosage suggestions of the drug. A judgment target: the user expects to make a certain judgment or decision. For example, the user inquires whether a certain drug is suitable for the treatment of a specific disease. A selection target: the user expects to select among multiple options. For example, the user inquires which of several drugs is more suitable for a certain symptom.

In this embodiment of the present disclosure, the drug information processing apparatus may recognize the target by analyzing keywords and semantics of the user input content. For example, the user input content is that the user inquires about the use, ingredients or side effects of the drug, and accordingly, the target may be classified as the information target; the user input content is to inquire whether the drug is suitable for a certain disease, and accordingly, the target may be classified as the judgment target.

In an optional implementation, the determining a target corresponding to the user input content based on the user input content includes: inputting the user input content into a target recognition model to obtain the target.

The target recognition model refers to a natural language processing model used to recognize user targets. Specifically, the target recognition model determines the target of the user by analyzing the user input content input by the user.

In this embodiment of the present disclosure, the drug information processing apparatus uses the target recognition model to classify the target of the user input content input by the user, so as to determine the target of the user. For example, the user input content is "usage and dosage of drug A", and the drug information processing apparatus analyzes it through the target recognition model and classifies it as an information query target (i.e., the target). In addition, the target recognition model may also limit its output form to keep consistency in art of speaking and expression of the generated text, while ensuring a unified and standardized format.

In this embodiment of the present disclosure, the drug information processing apparatus uses the user input content as input of the target recognition model, and uses the target recognition model to analyze features such as keywords, context and grammatical structure of the user input content, so as to determine the target. The target recognition helps to accurately match user needs and generate answers that conform to user targets, thereby effectively improving the accuracy of drug information query.

Step 104: determining a prompt based on the search result and the target, and inputting the prompt into a pre-trained drug processing model to obtain an output result corresponding to the user input content.

The prompt refers to a natural language text generated by embedding the search result and the target into a preset prompt template. The preset prompt template is a pre-designed text pattern with a specific structure and format, which is used to guide and standardize search behavior in the drug information processing process, and help users obtain required information more accurately and efficiently. Specifically, the preset prompt template usually consists of a fixed part and replaceable variables or placeholders. The fixed part defines the basic logic, field scope, task type, etc., of the search; the variable part is filled according to specific search needs.

The pre-trained drug processing model refers to a model that processes the prompt to obtain the output result corresponding to the user input content. Specifically, the pre-trained drug processing model may be a large language model (LLM). The large language model is a natural language-based processing model. Through the large language model, language rules and structures may be automatically learned, language meanings may be understood, and coherent text with correct grammar and semantics may be generated according to the understood meanings. The output result refers to an accurate answer to the user's inquiry about the user input content.

In this embodiment of the present disclosure, after acquiring the search result and the target, the search result and the target are combined with the preset prompt template. For example, the search result and the target are filled into corresponding positions of the preset prompt template by filling, to generate the prompt. Then, the prompt is input into the pre-trained drug processing model, and processed by the pre-trained drug processing model to obtain the output result corresponding to the user input content. It may be seen that the pre-trained drug processing model uses target recognition and generation planning capabilities to finally generate the output result that may conform to the user target.

Step 105: displaying the output result.

In this embodiment of the present disclosure, after obtaining the output result, the drug information processing apparatus displays the output result in a structured content presentation style. As shown in FIG. 5, the output result is displayed in an area 501 in FIG. 5 in the structured content presentation style, for example, drug A is a xxxx, clinical application: xxxx, adverse reaction: xxxx; drug A has significant efficacy in the treatment of various infectious diseases, and attention should be paid to contraindications and adverse reactions when using.

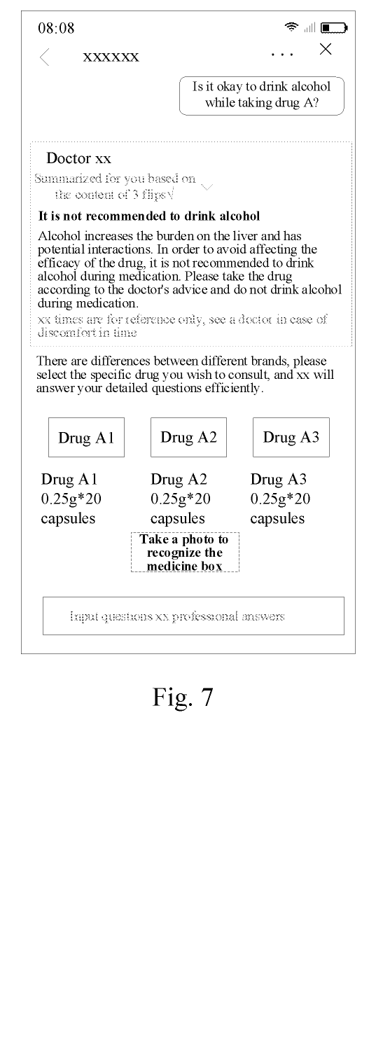

Exemplarily, FIG. 7 is a schematic diagram of a fifth drug information page provided by at least one embodiment of the present disclosure. As shown in FIG. 7, when the user input content is "is it okay to drink alcohol while taking drug A", after processing by the drug information processing system, the output result is returned, that is, it is not recommended to drink alcohol. Alcohol increases the burden on the liver and has potential interactions. In order to avoid affecting the efficacy of the drug, it is not recommended to drink alcohol during medication. Please take the drug according to the doctor's advice and do not drink alcohol during medication.

It should be noted that if the user input content is that the user only inputs drug A, the drug information processing apparatus may retrieve the entire instructions of drug A, and then summarize the instructions to obtain the output result.

In an optional implementation, displaying the output result includes: acquiring a first drug instruction corresponding to the search result, and displaying the output result and an instruction entry control of the first drug instruction.

The instruction entry control refers to a function control used to trigger viewing of the original text of the instruction, and the instruction entry control may be in any shape.

In this embodiment of the present disclosure, the drug information processing apparatus acquires the first drug instruction corresponding to the search result, and displays the instruction entry control of the first drug instruction below the output result.

As shown in FIG. 5, the instruction entry control of the first drug instruction is displayed in an area 502 below the output result, and is used to trigger entry into the display page of the first drug instruction, so that the user may conveniently view the original text of the first drug instruction.

In an optional implementation, the drug information processing method may further include: displaying a first drug instruction in response to a trigger operation on an instruction entry control.

The trigger operation on the instruction entry control may include a gesture operation (such as clicking, long pressing, double-clicking, etc.) on the instruction entry control, a voice control operation, an expression control operation, etc.

In this embodiment of the present disclosure, after receiving the trigger operation of the user on the instruction entry control, the drug information processing apparatus displays the first drug instruction. In this way, the user may conveniently view the original text of the first drug instruction, which may meet the requirements when the user needs to acquire more drug information, and is more in line with the actual needs of the user.

The drug information processing method provided by this embodiment of the present disclosure includes: acquiring user input content; performing search in a drug knowledge base based on the user input content to obtain a search result; determining a target corresponding to the user input content based on the user input content; determining a prompt based on the search result and the target, and inputting the prompt into a pre-trained drug processing model to obtain an output result corresponding to the user input content; and displaying the output result. With the above technical solution, for the acquired user input content, the corresponding search result is retrieved from the drug knowledge base, and the target is determined according to the user input content. Then, the pre-trained drug processing model is used to process the prompt determined based on the search result and the target to obtain the output result. By using the search enhanced generation technology, the user may obtain a more accurate output result without having high professional knowledge, which significantly improves the convenience of information query. Moreover, target recognition helps to accurately match user needs and generate an output result that conforms to the user target, thereby effectively improving the accuracy of drug information query.

In some embodiments, the drug information processing method may further include: displaying at least one associated drug question associated with the search result, and acquiring a first associated drug answer corresponding to a first associated drug question in response to a trigger operation on the first associated drug question in the at least one associated drug question.

The associated drug question is a drug question related to the search result. The first associated drug question refers to any associated drug question in the at least one associated drug question associated with the search result. The trigger operation on the first associated drug question may include a gesture operation (such as clicking, long pressing, double-clicking, etc.) on the first associated drug question, a voice control operation, an expression control operation, etc. The first associated drug answer refers to an accurate answer to the user's inquiry about the first associated drug question.

Specifically, the first associated drug answer is acquired in the following manner: after displaying the at least one associated drug question associated with the search result, the drug information processing apparatus receives the trigger operation on the first associated drug question in the at least one associated drug question, takes the first associated drug question as new user input content, and obtains the first associated drug answer corresponding to the first associated drug question with reference to steps 101 to 105. By using the search enhanced generation technology, the user may obtain a more accurate answer to the drug question without having high professional knowledge, which significantly improves the convenience of information query. Moreover, target recognition helps to accurately match user needs and generate an answer that conforms to the user target, thereby effectively improving the accuracy of drug information query.

In an optional implementation, the at least one associated drug question is displayed in a list manner or an option manner.

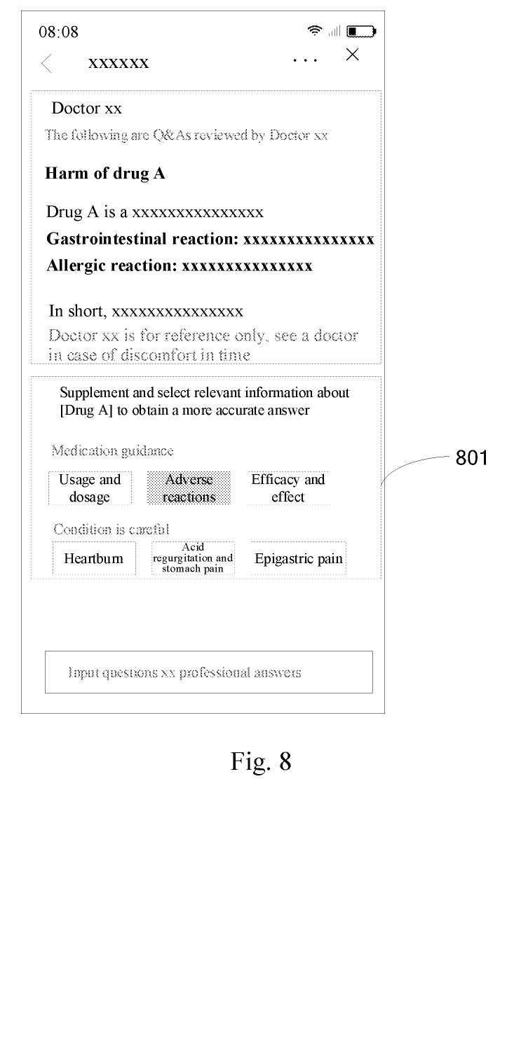

The list manner refers to an expression manner in which information is arranged in a list form. As shown in an area 503 in FIG. 5, associated drug questions are displayed in the list manner. The option manner refers to an expression manner in which a user is allowed to select from multiple alternative contents. As shown in an area 801 in FIG. 8, the associated drug questions are displayed in the option manner. FIG. 8 is a schematic diagram of a sixth drug information page provided by at least one embodiment of the present disclosure.

In this embodiment of the present disclosure, the drug information processing apparatus may display the at least one associated drug question associated with the search result below the search result, and acquire the first associated drug answer corresponding to the first associated drug question after receiving the trigger operation on the first associated drug question in the at least one associated drug question. This may effectively help the user to obtain the at least one associated drug question associated with the first drug while querying the first drug. When the user clicks on a specific associated drug question, the drug information processing apparatus may further display the corresponding first associated drug answer. This interaction manner not only improves the efficiency of information acquisition, but also enhances the convenience and comprehensiveness of user experience.

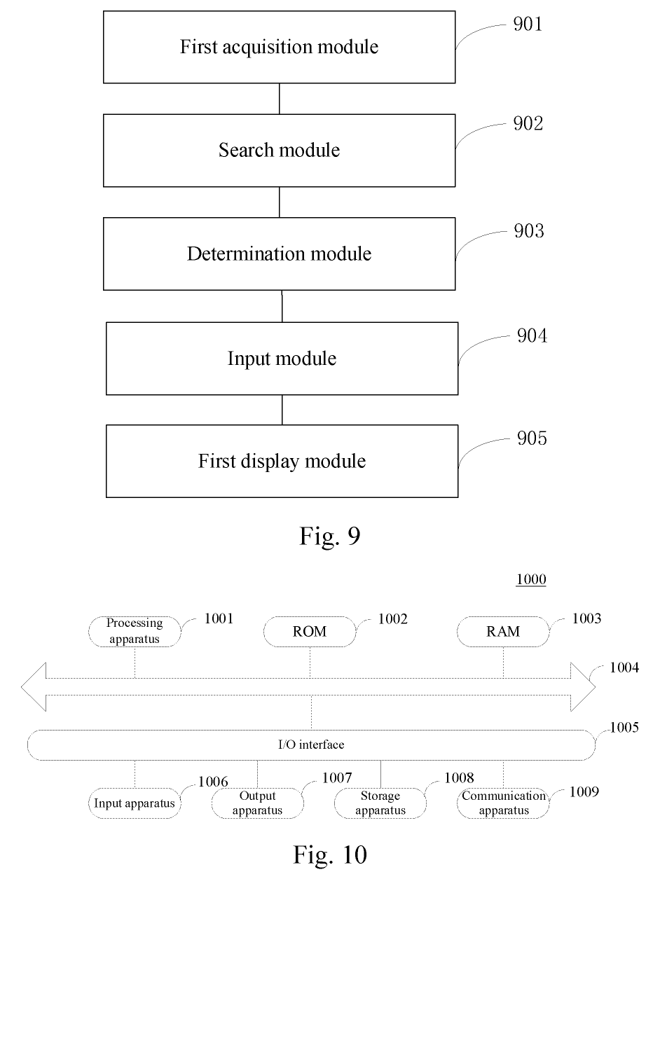

FIG. 9 is a schematic diagram of a structure of a drug information processing apparatus provided by at least one embodiment of the present disclosure. The apparatus may be implemented by software and/or hardware, and may generally be integrated in an electronic device. As shown in FIG. 9, the apparatus includes:

a first acquisition module 901 configured to acquire user input content;

a search module 902 configured to perform search in a drug knowledge base based on the user input content to obtain a search result;

a determination module 903 configured to determine a target corresponding to the user input content based on the user input content;

an input module 904 configured to determine a prompt based on the search result and the target, and input the prompt into a pre-trained drug processing model to obtain an output result corresponding to the user input content; and

a first display module 905 configured to display the output result.

In an optional implementation, the first acquisition module 901 includes at least one selected from the group consisting of:

a first acquisition sub-module configured to acquire the user input content in response to an input operation in a search box;

a second acquisition sub-module configured to acquire a first drug image in response to a trigger operation on a photographing recognition control, and obtain the user input content based on recognizing the first drug image; and

a third acquisition sub-module configured to acquire the user input content in response to a trigger operation on a drug consultation entry control in a drug associated page.

In an optional implementation, the drug consultation entry control includes at least one piece of second drug information associated with first drug information displayed on the drug associated page, and the third acquisition sub-module includes:

a first determination sub-module configured to determine a first drug name corresponding to the first drug information as the user input content if a trigger position of the trigger operation on the drug consultation entry control is in a non-information position; and

a second determination sub-module configured to determine a third drug name corresponding to third drug information as the user input content if the trigger position of the trigger operation on the drug consultation entry control is in the third drug information in the at least one piece of second drug information.

In an optional implementation, the search module 902 includes:

a search sub-module configured to perform search in the drug knowledge base by a keyword search manner and a semantic search manner based on the user input content, to acquire a second drug name and a first drug content segment, and determine the second drug name and the first drug content segment as the search result.

In an optional implementation, the determination module 903 is further configured to:

input the user input content into a target recognition model to obtain the target.

In an optional implementation, the first display module 905 includes:

a fourth acquisition sub-module configured to acquire a first drug instruction corresponding to the search result; and

a display sub-module configured to display the output result and an instruction entry control of the first drug instruction.

In an optional implementation, the apparatus further includes:

a second display module configured to display the first drug instruction in response to a trigger operation on the instruction entry control.

In an optional implementation, the apparatus further includes:

a third display module configured to display at least one associated drug question associated with the search result; and

a second acquisition module configured to acquire a first associated drug answer corresponding to a first associated drug question in response to a trigger operation on the first associated drug question in the at least one associated drug question.

In an optional implementation, the at least one associated drug question is displayed in a list manner or an option manner.

In an optional implementation, the apparatus further includes:

a third acquisition module configured to acquire drug information of a plurality of drugs; and

a construction module configured to construct the drug knowledge base based on the drug information of the plurality of drugs, where the drug knowledge base includes structured data and unstructured data.

The drug information processing apparatus provided by this embodiment of the present disclosure may perform the drug information processing method provided by any one of the embodiments of the present disclosure, and has corresponding functional modules for performing the method and beneficial effects.

At least one embodiment of the present disclosure further provides a non-transitory computer program product, including a computer program/instruction, where the drug information processing method provided by any one of the embodiments of the present disclosure is implemented when the computer program/instruction is executed by a processor.

FIG. 10 is a schematic diagram of a structure of an electronic device provided by at least one embodiment of the present disclosure.

Reference is made specifically to FIG. 10 below, which is a schematic diagram of a structure of an electronic device 1000 suitable for implementing the embodiments of the present disclosure. The electronic device 1000 in this embodiment of the present disclosure may include, but is not limited to, mobile terminals such as a mobile phone, a notebook computer, a digital broadcast receiver, a PDA (personal digital assistant), a PAD (tablet computer), a PMP (portable multimedia player), a vehicle-mounted terminal (e.g., a vehicle navigation terminal), etc., and fixed terminals such as a digital TV, a desktop computer, etc. The electronic device shown in FIG. 10 is only an example, and should not impose any limitation on the function and scope of use of the embodiments of the present disclosure.

As shown in FIG. 10, the electronic device 1000 may include a processing apparatus (e.g., a central processing unit, a graphics processor, etc.) 1001, which may perform various appropriate actions and processing according to a program stored in a read-only memory (ROM) 1002 or a program loaded from a storage apparatus 1008 into a random access memory (RAM) 1003. The RAM 1003 further stores various programs and data required for the operation of the electronic device 1000. The processing apparatus 1001, the ROM 1002 and the RAM 1003 are connected to each other through a bus 1004. An input/output (I/O) interface 1005 is also connected to the bus 1004.

Usually, the following apparatuses may be connected to the I/O interface 1005: an input apparatus 1006 including, for example, a touch screen, a touchpad, a keyboard, a mouse, a camera, a microphone, an accelerometer, a gyroscope, etc.; an output apparatus 1007 including, for example, a liquid crystal display (LCD), a speaker, a vibrator, etc.; a storage apparatus 1008 including, for example, a magnetic tape, a hard disk, etc.; and a communication apparatus 1009. The communication apparatus 1009 may allow the electronic device 1000 to perform wireless or wired communication with other devices to exchange data. Although FIG. 10 shows the electronic device 1000 having various apparatuses, it should be understood that not all the apparatuses shown here need to be implemented or provided. Alternatively, more or fewer apparatuses may be implemented or provided.

In particular, according to the embodiments of the present disclosure, the process described above with reference to the flowchart may be implemented as a computer software program. For example, this embodiment of the present disclosure includes a computer program product, which includes a computer program carried on a non-transitory computer-readable medium, where the computer program contains program code for performing the method shown in the flowchart. In such an embodiment, the computer program may be downloaded and installed from a network through the communication apparatus 1009, or installed from the storage apparatus 1008, or installed from the ROM 1002. When the computer program is executed by the processing apparatus 1001, the preceding functions limited in the drug information processing method of the embodiments of the present disclosure are executed.

It should be noted that the preceding computer-readable medium in the present disclosure may be a computer-readable signal medium, a computer-readable storage medium, or any combination thereof. The computer-readable storage medium may be, for example, but is not limited to, an electrical, magnetic, optical, electromagnetic, infrared, or semiconductor system, apparatus, device, or any combination thereof. More specific examples of the computer-readable storage medium may include, but are not limited to: an electrical connection having one or more wires, a portable computer magnetic disk, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination thereof. In the present disclosure, the computer-readable storage medium may be any tangible medium that contains or stores a program, which may be used by or used in combination with an instruction execution system, apparatus or device. In the present disclosure, the computer-readable signal medium may include a data signal propagated in a baseband or as a part of a carrier, and computer-readable program code is carried therein. The data signal propagated in this manner may be in multiple forms, and includes, but is not limited to, an electromagnetic signal, an optical signal, or any suitable combination thereof. The computer-readable signal medium may also be any computer-readable medium other than the computer-readable storage medium, and the computer-readable signal medium may send, propagate, or transmit the program used by or in combination with the instruction execution system, apparatus, or device. The program code contained on the computer-readable medium may be transmitted in any suitable medium, including, but not limited to: a wire, an optical cable, a radio frequency (RF), etc., or any suitable combination thereof.

In some implementations, clients and servers may communicate using any currently known or future developed network protocol, such as the hypertext transfer protocol (HTTP), and may be interconnected with any form or medium of digital data communication (for example, a communication network). Examples of the communication network include a local area network ("LAN"), a wide area network ("WAN"), an internet (for example, the Internet), a peer-to-peer network (for example, an Ad-Hoc network), and any network currently known or to be developed in the future.

The preceding computer-readable medium may be included in the preceding electronic device, or may exist alone without being assembled into the electronic device.

The above-mentioned computer-readable medium carries one or more programs, and when the above-mentioned one or more programs are executed by the electronic device, the electronic device: acquires user input content; performs search in a drug knowledge base based on the user input content to obtain a search result; determines a target corresponding to the user input content based on the user input content; determines a prompt based on the search result and the target, and inputs the prompt into a pre-trained drug processing model to obtain an output result corresponding to the user input content; and displays the output result.

Computer program code for performing the operations of the present disclosure may be written in one or more programming languages or a combination thereof, where the preceding programming languages include, but are not limited to, object-oriented programming languages such as Java, Smalltalk, and C++, and further include conventional procedural programming languages such as "C" language or similar programming languages. The program code may be completely executed on a computer of a user, partially executed on a computer of a user, executed as an independent software package, partially executed on a computer of a user and partially executed on a remote computer, or completely executed on a remote computer or a server. In the scenario involving the remote computer, the remote computer may be connected to the computer of the user through any kind of network, including a local area network (LAN) or a wide area network (WAN), or may be connected to an external computer (for example, connected through the Internet with the aid of an Internet service provider).

The flowchart and block diagrams in the drawings illustrate the possibly implemented architectures, functions, and operations of the system, the method, and the computer program product according to various embodiments of the present disclosure. In this regard, each block in the flowchart or block diagram may represent a module, program segment, or part of code, which contains one or more executable instructions for implementing the specified logical functions. It should also be noted that, in some alternative implementations, the functions marked in the blocks may also occur in an order different from that marked in the drawings. For example, two blocks shown in succession may actually be performed substantially in parallel, or they may sometimes be performed in the reverse order, depending on the functions involved. It should also be noted that each block in the block diagram and/or the flowchart, and a combination of the blocks in the block diagram and/or the flowchart may be implemented by a dedicated hardware-based system that executes specified functions or operations, or may be implemented by a combination of dedicated hardware and computer instructions.

The units involved in the embodiments of the present disclosure may be implemented in a software manner, and may also be implemented in a hardware manner. The name of a unit does not constitute a limitation on the unit under certain circumstances.

The functions described herein may be performed at least in part by one or more hardware logic components. For example, without limitation, exemplary types of hardware logic components that may be used include: a field programmable gate array (FPGA), an application specific integrated circuit (ASIC), an application specific standard product (ASSP), a system on chip (SOC), a complex programmable logical device (CPLD), etc.

In the context of the present disclosure, a machine-readable medium may be a tangible medium that may contain or store a program for use by or in combination with an instruction execution system, apparatus or device. The machine-readable medium may be a machine-readable signal medium or a machine-readable storage medium. The machine-readable medium may include, but is not limited to, an electronic, magnetic, optical, electromagnetic, infrared, or semiconductor system, apparatus or device, or any suitable combination of the preceding. More specific examples of the machine-readable storage medium may include an electrical connection based on one or more wires, a portable computer magnetic disk, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination of the preceding.

It may be understood that before using the technical solutions disclosed in the embodiments of the present disclosure, the user should be informed of the type, scope of use, use scenarios, etc., of the information involved in the present disclosure in an appropriate manner in accordance with relevant laws and regulations, and the authorization of the user should be obtained.

The preceding description is only a preferred embodiment of the present disclosure and an explanation of the technical principles employed. Those skilled in the art should understand that the scope of disclosure involved in the present disclosure is not limited to the technical solutions formed by the specific combination of the preceding technical features, and should also cover other technical solutions formed by any combination of the preceding technical features or their equivalent features without departing from the preceding concept of disclosure. For example, the preceding features and the technical features disclosed in the present disclosure (but not limited to) with similar functions are replaced each other to form a technical solution.

Additionally, although the operations have been described in a particular order, it should not be understood as requiring that such operations are performed in the particular order shown or in a sequential order. Under certain circumstances, multitasking and parallel processing may be advantageous. Likewise, although the preceding discussion contains several implementation-specific details, these should not be construed as limitations on the scope of the present disclosure. Certain features that are described in the context of separate embodiments may also be implemented in combination in a single embodiment. Conversely, various features described in the context of a single embodiment may also be implemented in multiple embodiments individually or in any suitable sub-combinations.

Although the subject matter has been described in language specific to structural features and/or logical actions of the method, it should be understood that the subject matter defined in the appended claims is not necessarily limited to the specific features or actions described above. Rather, the specific features and actions described above are merely exemplary forms of implementing the claims.

1. A resource information processing method, comprising:
acquiring user input content;
performing search in a resource knowledge base based on the user input content to obtain a search result;
determining a target corresponding to the user input content based on the user input content;
determining a prompt based on the search result and the target, and inputting the prompt into a pre-trained resource processing model to obtain an output result corresponding to the user input content; and
displaying the output result.

2. The method of claim 1, wherein the acquiring user input content comprises at least one selected from the group consisting of:
acquiring the user input content in response to an input operation in a search box;
acquiring a first resource image in response to a trigger operation on a photographing recognition control, and obtaining the user input content based on recognizing the first resource image; and
acquiring the user input content in response to a trigger operation on a resource consultation entry control in a resource associated page.

3. The method of claim 2, wherein the resource consultation entry control comprises at least one piece of second resource information associated with first resource information displayed on the resource associated page, and the acquiring the user input content in response to a trigger operation on a resource consultation entry control in a resource associated page comprises:
in response to a trigger position of the trigger operation on the resource consultation entry control being in a non-information position, determining a first resource name corresponding to the first resource information as the user input content; and
in response to the trigger position of the trigger operation on the resource consultation entry control being in third resource information in the at least one piece of second resource information, determining a third resource name corresponding to the third resource information as the user input content.

4. The method of claim 1, wherein the performing search in a resource knowledge base based on the user input content to obtain a search result comprises:
performing search in the resource knowledge base by a keyword search manner and a semantic search manner based on the user input content, to acquire a second resource name and a first resource content segment, and determine the second resource name and the first resource content segment as the search result.

5. The method of claim 1, wherein the determining a target corresponding to the user input content based on the user input content comprises:
inputting the user input content into a target recognition model to obtain the target.

6. The method of claim 1, wherein the displaying the output result comprises:
acquiring a first resource instruction corresponding to the output result; and
displaying the output result and an instruction entry control of the first resource instruction.

7. The method of claim 6, further comprising:
displaying the first resource instruction in response to a trigger operation on the instruction entry control.

8. The method of claim 1, further comprising:
displaying at least one associated resource question associated with the output result; and
acquiring a first associated resource answer corresponding to a first associated resource question in response to a trigger operation on the first associated resource question in the at least one associated resource question.

9. The method of claim 8, wherein the at least one associated resource question is displayed in a list manner or an option manner.

10. The method of claim 1, further comprising:
acquiring resource information of a plurality of resources; and
constructing the resource knowledge base based on the resource information of the plurality of resources, wherein the resource knowledge base comprises structured data and unstructured data.

11. An electronic device, comprising:
a processor; and
a memory configured to store executable instructions executable by the processor, wherein
the processor is configured to read the executable instructions from the memory, and execute the executable instructions to implement a resource information processing method, and the resource information processing method comprises:
acquiring user input content;
performing search in a resource knowledge base based on the user input content to obtain a search result;
determining a target corresponding to the user input content based on the user input content;
determining a prompt based on the search result and the target, and inputting the prompt into a pre-trained resource processing model to obtain an output result corresponding to the user input content; and
displaying the output result.

12. The electronic device of claim 11, wherein the acquiring user input content comprises at least one selected from the group consisting of:
acquiring the user input content in response to an input operation in a search box;
acquiring a first resource image in response to a trigger operation on a photographing recognition control, and obtaining the user input content based on recognizing the first resource image; and
acquiring the user input content in response to a trigger operation on a resource consultation entry control in a resource associated page.

13. The electronic device of claim 12, wherein the resource consultation entry control comprises at least one piece of second resource information associated with first resource information displayed on the resource associated page, and the acquiring the user input content in response to a trigger operation on a resource consultation entry control in a resource associated page comprises:
in response to a trigger position of the trigger operation on the resource consultation entry control being in a non-information position, determining a first resource name corresponding to the first resource information as the user input content; and
in response to the trigger position of the trigger operation on the resource consultation entry control being in third resource information in the at least one piece of second resource information, determining a third resource name corresponding to the third resource information as the user input content.

14. The electronic device of claim 11, wherein the performing search in a resource knowledge base based on the user input content to obtain a search result comprises:
performing search in the resource knowledge base by a keyword search manner and a semantic search manner based on the user input content, to acquire a second resource name and a first resource content segment, and determine the second resource name and the first resource content segment as the search result.

15. The electronic device of claim 11, wherein the determining a target corresponding to the user input content based on the user input content comprises:
inputting the user input content into a target recognition model to obtain the target.

16. The electronic device of claim 11, wherein the displaying the output result comprises:
acquiring a first resource instruction corresponding to the output result; and
displaying the output result and an instruction entry control of the first resource instruction.

17. The electronic device of claim 16, wherein the method further comprises:
displaying the first resource instruction in response to a trigger operation on the instruction entry control.

18. The electronic device of claim 11, wherein the method further comprises:
displaying at least one associated resource question associated with the output result; and
acquiring a first associated resource answer corresponding to a first associated resource question in response to a trigger operation on the first associated resource question in the at least one associated resource question.

19. The electronic device of claim 18, wherein the at least one associated resource question is displayed in a list manner or an option manner.

20. A non-transitory computer-readable storage medium, wherein the non-transitory computer-readable storage medium stores a computer program, and the computer program is executed by a processor to cause the processor to perform a resource information processing method, and the resource information processing method comprises:
acquiring user input content;
performing search in a resource knowledge base based on the user input content to obtain a search result;
determining a target corresponding to the user input content based on the user input content;
determining a prompt based on the search result and the target, and inputting the prompt into a pre-trained resource processing model to obtain an output result corresponding to the user input content; and
displaying the output result.
